(19) United States
(12) Patent Application Publication
(10) Pub. No.: US 2025/0057799 A1
(43) Pub. Date: Feb. 20, 2025

(54) Anti-Inflammatory Methods with Compositions Comprising Site-Specific Isotopically-Modified Fatty Acids

(71) Applicant: BioJiva LLC, San Jose, CA (US)

(72) Inventor: Mikhail S. Shchepinov, Oxford (GB)

(73) Assignee: BioJiva LLC, BioJiva LLC, CA (US)

(21) Appl. No.: 18/722,949

(22) PCT Filed: Dec. 21, 2022
(86) PCT No.: PCT/US2022/053687

§ 371 Date: Jun. 21, 2024

Related U.S. Application Data
(60) Provisional application No. 63/293,208, filed on Dec. 23, 2021.
(60) Provisional application No. 63/292,388, filed on Dec. 21, 2021.

(51) Int. Cl. A61K 31/202; A61P 11/00; A61P 19/02; A61P 29/00

(52) U.S. Cl. CPC A61K 31/202; A61P 11/00; A61P 19/02; A61P 29/00

(57) ABSTRACT

Disclosed herein are methods of treating or inhibiting the progression of inflammatory diseases, comprising administering an effective amount of a polyunsaturated substance to a patient having inflammation and in need of treatment, wherein the polyunsaturated substance is chemically modified such that one or more bonds are stabilized against oxidation. More specifically, the present disclosure provides methods for treating, reducing, or preventing inflammatory conditions by administering to a subject compositions comprising essential polyunsaturated fatty acids (PUFAs) and derivatives thereof having site-specific isotopic modifications at one or more “bis-allylic” (a CH₂ group found between two alkene fragments) and/or at one or more “pro-bis-allylic” (a methylene group that become a bis-allylic position upon desaturation) positions.

### CROSS-REFERENCE

This application claims the benefit of U.S. Provisional Application No. 63/292,388 filed December 21,2021; and U.S. Provisional Application No. 63/293,208 filed Dec. 23, 2021, which are incorporated herein by reference in their entirety.

### BACKGROUND

Oxidative damage is implicated in a wide variety of diseases such as mitochondrial diseases, neurodegenerative diseases, neurodegenerative muscle diseases, retinal diseases, energy processing disorders, kidney diseases, hepatic diseases, lipidemias, cardiac diseases, inflammation, and genetic disorders.

While the number of diseases associated with oxidative stress are numerous and diverse, it is well established that oxidative stress is caused by disturbances to the normal redox state within cells. An imbalance between routine production and detoxification of reactive oxygen species (“ROS”) such as peroxides and free radicals can result in oxidative damage to cellular structures and machinery. Under normal conditions, a potentially important source of ROS in aerobic organisms is the leakage of activated oxygen from mitochondria during normal oxidative respiration. Additionally, it is known that macrophages and enzymatic reactions also contribute to the generation of ROS within cells. Because cells and their internal organelles are lipid membrane-bound, ROS can readily contact membrane constituents and cause lipid oxidation. Ultimately, such oxidative damage can be relayed to other biomolecules within the cell, such as DNA and proteins, through direct and indirect contact with activated oxygen, oxidized membrane constituents, or other oxidized cellular components. Thus, one can readily envision how oxidative damage may propagate throughout a cell give the mobility of internal constituents and the interconnectedness of cellular pathways.

PUFAs endow mitochondrial membranes with appropriate fluidity necessary for optimal oxidative phosphorylation performance. PUFAs also play an important role in initiation and propagation of the oxidative stress. PUFAs react with ROS through a chain reaction that amplifies an original event (Sun M, Salomon R G, J. Am. Chem. Soc. 2004; 126:5699-5708). However, nonenzymatic formation of high levels of lipid hydroperoxidesis known to result in several detrimental changes. Indeed, Coenzyme Q10 has been linked to increased PUFA toxicity via PUFA peroxidation and the toxicity of the resulting products (Do T Q et al., PNAS USA 1996; 93:7534-7539). Such oxidized products negatively affect the fluidity and permeability of their membranes; they lead to oxidation of membrane proteins; and they can be converted into a large number of highly reactive carbonyl compounds. The latter include reactive species such as acrolein, malonic dialdehyde, glyoxal, methylglyoxal, etc. (Negre-Salvayre A, et al., Brit. J. Pharmacol. 2008; 153:6-20). But the most prominent products of PUFA oxidation are alpha, beta-unsaturated aldehydes such as 4-hydroxynon-2-enal (4-HNE; formed from n-6 PUFAs like LA or AA), 4-hydroxyhex-2-enal (4-HHE; formed from n-3 PUFAs like ALA or DHA), and corresponding ketoaldehydes (Esterfbauer H, et al., Free Rad. Biol. Med. 1991; 11:81-128; Long E K, Picklo M J. Free Rad. Biol. Med. 2010; 49:1-8). These reactive carbonyls cross-link (bio)molecules through Michael addition or Schiff base formation pathways and have been implicated in a large number of pathological processes (such as those introduced above), age-related and oxidative stress-related conditions, and aging. Importantly, in some cases, PUFAs appear to oxidize at specific sites because methylene groups of 1,4-diene systems (the bis-allylic position) are substantially less stable to ROS, and to enzymes such as cyclooxygenases, cytochromes and lipoxygenases, than allylic methylenes.

Eicosanoids are important cell signaling molecules involved in an array of physiological and pathological processes, including various immune responses such as promoting inflammation, inhibiting inflammation, allergy, and fever. There are multiple oxidative and immune response related pathways in the biosynthesis of eicosanoids from arachidonic acid.

In one pathway, the enzyme 5-lipoxygenase (5-LO or ALOX5) converts arachidonic acid (ARA) into 5-hydroperoxyeicosatetraenoic acid (5-HPETE) and leukotriene A4 (LTA4), which is then converted into different leukotrienes (LTC4, LTD4, and LTE4) by downstream enzymes. LTC4, LTD4, and LTE4 are potent bronchoconstrictors and stimulators of mucus secretion in lung tissue and are known to promote hypersensitivity reactions in respiratory conditions such as asthma.

In another pathway leading to oxidative and inflammatory responses, 15-lipoxygenase (15-lipoxygenase 1, 15-LOX, 15-LOX1, or ALOX15) and 12-lipoxygenase (12-LOX or ALOX12) metabolize arachidonic acid to form 15(S)-hydroperoxyeicosatetraenoic acid (15(S)-HPETE) and 12(S)-hydroperoxyeicosatetraenoic acid (12(S)-HPETE). Both 15(S)-HPETE and 12(S)-HPETE are further reduced by cellular glutathione peroxidase to their corresponding hydroxy analogs, 15-hydroxyicosatetraenoic acid (15(S)-HETE) and 12-hydroxyeicosatetraenoic acid (12(S)-HETE), respectively. 15(S)-HPETE and 15(S)-HETE are further metabolized to various bioactive products such as lipoxins, hepoxillins, eoxins, 8(S),15(S)-diHETE, 5(S),15(S)-diHETE and 15-oxo-eicosatetraenoic acid (15-oxo-ETE). The 12/15-lipoxygenase metabolites have been shown to have both pro- and anti-inflammatory properties.

In yet another pathway, COX1 and COX2 (also known as prostaglandin-endoperoxide synthase-1 (PTGS1) and PTGS2, respectively) initiates metabolization of arachidonic acid to a subclass of eicosanoids that includes prostaglandins (such as prostacyclins), and thromboxanes. Prostaglandins are mediators of inflammatory and anaphylactic reactions and thromboxanes are mediators of vasoconstriction and platelet aggregation, playing a major role in blood clotting.

In another pathway, cytochrome P450 enzymes metabolize arachidonic acid to EETs (epoxyeicosatrienoic acids). EETs can promote the active termination of inflammation through mediating a broad array of anti-inflammatory and pro-resolving mechanisms, including mitigation of the cytokine storm.

Although elevated cytokine levels in COVID-19 have been identified as a major factor contributing to morbidity and mortality, the role of eicosanoids in COVID-19 as key mediators of both inflammation and thrombosis and their active resolution remains poorly characterized.

Severe Acute Respiratory Syndrome Coronavirus 2 (SARS-CoV-2) is related to SARS-CoV and several SARS-like bat CoVs (F. Wu, et al., A new coronavirus associated with human respiratory disease in China. Nature 579, 265-269 (2020)). SARS-CoV-2 is the third coronavirus to cause severe respiratory illness in humans, called coronavirus disease 2019 (COVID-19).

The factors that trigger severe illness in individuals infected with SARS-CoV-2 are not completely understood. However, an excessive inflammatory response to SARS-CoV-2 is thought to be a major cause of disease severity and death in patients with COVID-19 and is associated with high levels of circulating cytokines, profound lymphopenia and substantial mononuclear cell infiltration in the lungs, heart, spleen, lymph nodes and kidney. (Mehta, P. et al., Lancet, 395, 1033-1034 (2020); Xu, Z. et al., Lancet Respir. Med., 8, 420-422(2020); Chen, Y. et al., Preprint at medRxiv athttps://doi.org/10.110112020.03.27.20045.427 (2020); Diao, B., et al., Preprint atmedRxiv htips://doi.org/10.1.101/2020.03.04.2.0031120 (2020).)

Almost all patients with COVID-19 present with lung involvement, as evidenced by chest radiography, whereas severe complications—such as ARDS (Acute Respiratory Disease Syndrome) and death—are only observed in a subgroup of patients. Higher levels of inflammatory markers in blood (including C-reactive protein, ferritin, and D-dimers), an increased neutrophil-to-lymphocyte ratio and increased serum levels of several inflammatory cytokines and chemokines have been associated with disease severity and death. The systemic cytokine profiles observed in patients with severe COVID-19 show similarities to those observed in cytokine release syndromes (or “cytokine storms”), such as macrophage activation syndrome, with increased production of cytokines such as IL-6, IL-7 and tumor necrosis factor (TNF) and also of inflammatory chemokines including CC-chemokine ligand 2 (CCL2), CCL3 and CXC-chemokine ligand 10 (CXCL10), as well as of the soluble form of the a-chain of the IL-2 receptor. (Merad, M. and Martin, J., Nature Reviews Immunology, 20, 355-362(2020).

One publication suggests that SARS-CoV-2 infection leads to the up-regulation of inflammatory enzymes, including COX-1 and COX-2, which subsequently produce eicosanoids, including prostaglandins (PGs), leukotrienes (LTs), and thromboxanes (TXs). (Hammock, B., et al., Eicosanoids The Overlooked Storm in Coronavirus Disease 2019 (COVID-19)?, Am. J. Pathol., (2020), https://doi.org/10.1016/j.aipath.2020.06.010.) The paper suggests these proinflammatory eicosanoids induce cytokine storms that mediate widespread inflammatory responses and organ damage in severe COVID-19 patients. The paper also suggests that EETs may counter-regulate the unabated systemic inflammatory response and organ failure associated with COVID-19 infection.

The immune system of younger people is less developed and so it usually produces lower levels of inflammation-driving cytokines. Separately, COVID patients demonstrate a remarkably high incidence of thrombotic complications (almost half of the patients admitted to the ICU), even with thromboprophylaxis, further contributing to the high mortality rate from COVID due to pulmonary embolism, strokes, and heart attacks. Abnormal blood clotting is observed in COVID patients, including clots in small blood vessels, deep vein thrombosis in the legs, clots in the lungs and stroke causing clots in cerebral arteries. It is well known that COVID-19-related blood clotting does not respond well even to high doses of blood thinners. (Magro, C., et al., Translational Res., 2020, 220:1-13.)

Prior studies have examined the relative enzymatic activity of cyclooxygenases and lipoxygenases upon isotopologues of arachidonic acid deuterated at bis-allylic positions, but these were mechanistic studies that did not use deuterated arachidonic acid in physiological or therapeutic amounts. One study tested the enzymatic activity of purified COX and LOX on different isotopologues of arachidonic acid, but the study did not test activity in cells. (Chistyakov, D., et al., Deuterated Arachidonic Acids Library for Regulation of Inflammation and Controlled Synthesis of Eicosanoids: An In Vitro Study, Molecules, 2018, 23:3331; which is hereby incorporated by reference in its entirety.) In another study, a macrophage cell-line was incubated with D-ARA or normal ARA, and cell-culture supernatants were analyzed for the amount of eicosanoids produced and secreted. However, this study used non-physiological conditions where arachidonic acid at a molar concentration of 25 micromolar was incubated with the cells for 24 hours, which resulted in incorporation of up to 90% of the arachidonic acid in cell membranes a percentage unattainable by several-fold under physiological conditions. (Navratil, A. et al., Lipidomics Reveals Dramatic Kinetic Isotope Effects during the Enzymatic Oxygenation of Polyunsaturated Fatty Acids Ex Vivo). Thus, neither study has shown whether compositions comprising bis-allylic isotopically modified essential PUFAs, including arachidonic acid, can inhibit inflammation in vivo, which is important given the multiple metabolic pathways involving arachidonic acid and the potential pro- and anti-inflammatory effects resulting therefrom. Further, the purified enzymatic study (Chistyakov et al) and the cell assay (Navratil et al) both used very high concentrations of the designated ARA species in the absence of other PUFAs and did not examine biologically relevant complex mixtures of fatty acids present at low concentrations, and enzymatic products derived therefrom, whether in in vitro or in vivo model systems.

Apart from the pro inflammatory enzymatic products of ARA oxidation, the second major element of inflammation is the non-enzymatic chain reaction of PUFA damage, known as lipid peroxidation (LPO), which may be initiated by enzymes or by small molecule ROS. This pro-inflammatory non-enzymatic component is impervious to antioxidant approaches due to the structural aspects of the lipid bilayers making up membranes Inflammation associated LPO can be initiated through various mechanisms. (Zhang et al, Myeloperoxidase Functions as a Major Enzymatic Catalyst for Initiation of Lipid Peroxidation at Sites of Inflammation, J Blot Chem 2002 doi: 10.1074/jbc.M209124200).

### SUMMARY

Generally, the present disclosure provides a method of treating or inhibiting the progression of inflammatory diseases, comprising administering an effective amount of a polyunsaturated substance to a patient having inflammation and in need of treatment, wherein the polyunsaturated substance is chemically modified such that one or more bonds are stabilized against oxidation, wherein the polyunsaturated substance or a polyunsaturated metabolite thereof comprising said one or more stabilized bonds is incorporated into the patient's body following administration.

More specifically, the present disclosure provides methods for treating, reducing, or preventing inflammatory conditions by administering to a subject compositions comprising essential polyunsaturated fatty acids (PUFAs) and derivatives thereof having site-specific isotopic modifications at one or more “bis-allylic” (a CH₂ group found between two alkene fragments) and/or at one or more “pro-bis-allylic” (a methylene group that become a bis-allylic position upon desaturation) positions. (See U.S. Pat. No. 10,477,304, “Site-Specific Isotopic Labeling of 1,4-Diene Systems,” for such isotopically-modified compounds and synthesis methods, which is hereby incorporated-by-reference in its entirety; see also U.S. Pat. Nos. 10,154,978; 10,058,612; 10,154,983; 10,058,522, each of which is hereby incorporated-by-reference in its entirety and at least for their description of bis-allylic and pro-bis-allylic isotopically modified PUFAs and mimetics thereof.)

In certain aspects, the present disclosure provides methods for preventing, treating, or reducing inflammatory disorders or conditions associated with increased levels in ROS and/or lipid peroxidation in cells by administering compositions comprising one or more species of bis-allylic isotopically modified essential PUFAs, including one or more omega-3 and omega-6 fatty acids and derivatives thereof such as esters thereof. The ester can be, for example, a triglyceride, diglyceride, or monoglyceride.

Inflammatory disorders or conditions associated with lipid oxidation due to reactive oxygen species include, but are not limited to, asthma, rheumatoid arthritis, juvenile chronic arthritis, osteoarthritis, myositis, Crohn's disease, gastritis, colitis, ulcerative colitis, inflammatory bowel disease, proctitis, pelvic inflammatory disease, systemic lupus erythematosus, rhinitis, conjunctivitis, scleritis, chronic inflammatory polyneuropathy, Lyme disease, psoriasis, dermatitis, eczema, autoimmune disorders, atherosclerosis, and COVID-19.

In certain aspects, the inflammatory disorders or conditions can be those associated with COVID-19 (SARS CoV-2), SARS CoV-1, MERS, and influenza. In another aspect, the inflammatory disorder or condition is hyperinflammation or a cytokine storm for COVID-19 patients. In one aspect, the inflammatory disorder or condition is inflammation of the lung or airways of COVID-19 patients. In another aspect, the inflammatory disorder or condition relates to thrombotic complications for COVID-19 patients. (See, e.g., Price, L., et al., Thrombosis and COVID-19 pneumonia: the clot thickens!. Eur Respir J 2020; in press (https://doi.org/10.1183/13993003.01608-2020).

In certain aspects, the isotopically modified PUFAs described herein, and esters and mixtures thereof, are administered to subjects with a thrombotic condition, including thrombotic complications due to pulmonary embolism, strokes, heart attacks, and abnormal blood clotting in COVID patients, including clots in small blood vessels, deep vein thrombosis in the legs, clots in the lungs and stroke causing clots in cerebral arteries. It is well known that COVID-19-related blood clotting does not respond well even to high doses of blood thinners. (Magro, C., et al., Translational Res., 2020,220:1-13.) In some aspects, 7,7,10,10,13,13-D6-arachidonic acid is administered for these thrombotic conditions.

Administered species (or combination of species) of bis-allylic isotopically modified essential PUFAs include, but are not limited to, linolenic acid, linoleic acid, arachidonic acid, eicosapentaenoic acid, docosahexaenoic acid and derivatives thereof, including esters and mimetics thereof. Exemplary esters include monoglycerides, diglycerides, and triglycerides. In some aspects of the present disclosure, the bis-allylic isotopically modified PUFAs include, for example, DC and/or deuterated and/or tritiated linoleic acid (LA), alpha linolenic acid (ALA), eicosapentaenoic acid (EPA), arachidonic acid (ARA) and docosahexaenoic acid (DHA), and derivatives thereof, including esters.

For each of these isotopically modified essential PUFA species (i.e., LA, ALA, ARA, EPA, and DHA), there is at least one ¹³C or deuterium or tritium at one bis-allylic position. In other aspects, for each of these species, the amount of isotopic modification ranges from at least one deuterium or tritium at one bis-allylic position to all hydrogens being substituted at all bis-allylic positions. Some non-limiting examples include the species encompassed by Formula (1) (see herein) such as: 11-D-linoleic acid; 11,11-D2-linoleic acid; 11-D-linolenic acid; 11,11-D2-linolenic acid; 14-D-linolenic acid; 14,14-D2-linolenic acid; 11,14-D2-linolenic acid; 11,11,14,14-D4-linolenic acid; 7,7-D2-arachidonic acid; 10,10-D2-arachidonic acid; 13,13-D2-arachidonic acid; 7,7,10,10-D4-arachidonic acid; 7,7,13,13-D4-arachidonic acid; 10,10,13,13-D4-arachidonic acid; 7,7,10,10,13,13-D6-arachidonic acid; 7,7-D2-eicosapentaenoic acid; 10,10-D2-eicosapentaenoic acid; 13,13-D2-eicosapentaenoic acid; 16,16-D2-eicosapentaenoic acid; 7,7,10,10-D4-eicosapentaenoic acid; 7,7,13,13-D4-eicosapentaenoic acid; 7,7,16,16-D4-eicosapentaenoic acid; 10,10,13,13-D4-eicosapentaenoic acid; 10,10,16,16-D4-eicosapentaenoic acid; 13,13,16,16-D4-eicosapentaenoic acid; 7,7,10,10,13,13-D6-eicosapentaenoic acid; 7,7,10,10,16,16-D6-eicosapentaenoic acid; 7,7,13,13,16,16-D6-eicosapentaenoic acid; 10,10,13,13,16,16-D6-eicosapentaenoic acid; 7,7,10,10,13,13,16,16-D8-eicosapentaenoic acid; 6,6-D2-docosahexaenoic acid; 9,9-D2-docosahexaenoic acid; 12,12-D2-docosahexaenoic acid; 15,15-D2-docosahexaenoic acid; 18,18-D2-docosahexaenoic acid; 6,6,9,9-D4-docosahexaenoic acid; 6,6,12,12-D4-docosahexaenoic acid; 6,6,15,15-D4-docosahexaenoic acid; 6,6,18,18-D4-docosahexaenoic acid; 9,9,12,12-D4-docosahexaenoic acid; 9,9,15,15-D4-docosahexaenoic acid; 9,9,18,18-D4-docosahexaenoic acid; 12,12,15,15-D4-docosahexaenoic acid; 12,12,18,18-D4-docosahexaenoic acid; 15,15,18,18-D4-docosahexaenoic acid; 6,6,9,9,12,12-D6-docosahexaenoic acid; 6,6,9,9,15,15-D6-docosahexaenoic acid; 6,6,9,9,18,18-D6-docosahexaenoic acid; 9,9,12,12,15,15-D6-docosahexaenoic acid; 9,9,12,12,18,18-D6-docosahexaenoic acid; 12,12,15,15,18,18-D6-docosahexaenoic acid; 6,6,15,15,18,18-D6-docosahexaenoic acid; 9,9,15,15,18,18-D6-docosahexaenoic acid; 6,6, 12,12,15,15-D6-docosahexaenoic acid; 6,6,12,12,18,18-D6-docosahexaenoic acid; 6,6,9,9,12,12,15,15-D8-docosahexaenoic acid; 6,6,9,9,12,12,18,18-D8-docosahexaenoic acid; 6,6,9,9,15,15,18,18-D8-docosahexaenoic acid; 6,6,12,12,15,15,18,18-D8-docosahexaenoic acid; 9,9,12,12,15,15,18,18-D8-docosahexaenoic acid; 6,6,9,9,12,12,15,15,18,18-D10-docosahexaenoic acid. For the above-listed species, the POSA understands the numbering convention, where numbers preceding “D” indicate the carbon position and the number following “D” indicates the total number of deuterium. Each of the positions in the above-listed species are the carbon positions of bis-allylic sites. With each of these non-limiting examples, the modified fatty acid can be in a derivative form, including an ester form. Further, with each of these non-limiting examples, the fatty acid can be further modified by isotopically modifying one or more bis-allylic carbons with ¹³C. Further, with each of these non-limiting examples, the fatty acid can be substituted with tritium instead of deuterium at the specified locations. Yet further, with each of these non-limiting examples, the fatty acid can be further modified at one or more pro-bis-allylic positions with ¹³C and/or deuterium or tritium. In related aspects, the administered compositions can comprise a mixture of two or more of the above-listed species.

As used herein, and unless stated otherwise, reference to a particular PUFA (e.g., LA, ALA, AA, EPA, or DHA) shall encompass acids and “derivatives,” which include for example esters (as well as thioesters and amides), mimetics, and pro-drugs. Further, as used herein, deuterated fatty acids and derivatives may be substituted with tritium, so long as the tritium replaces the deuterium at the same position(s). Additional stabilization of the bis-allylic position could also include replacement of one or more of bis-allylic carbon atoms with a heavy isotope, alone or in conjunction with the deuteration (or tritiation), as the isotope effect (IE) resulting in stabilization of a bond with heavy isotopes is additive per long-established and fundamental chemical principles. (Westheimer, Chem. Rev. (1961), 61:265-273; Shchepinov, Rejuvenation Res., (2007), 10:47-59; Hill et al., Free Radic. Biol. Med., (2012), 53:893-906; Andreyev et al., Free Radic. Biol. Med., (2015), 82:63-72. Bigeleisen, J. The validity of the use of tracers to follow chemical reactions. Science, (1949), 110:14-16.)

In other aspects, bis-allylic isotopically modified LA, ALA, EPA, and/or DHA must be administered in combination with one or more of isotopically-modified arachidonic species as described herein. Stabilized, i.e., bis-allylic isotopically-modified AA, is expected to be especially effective for inflammation pathways based on the well-established metabolic pathway where oxidized AA is specifically involved in damaging species such as cyclooxygenase (COX) mediated thromboxane and prostacyclin formation-which is directly related to inflammation.

For aspects that comprise the administration of bis-allylic modified arachidonic acid and its derivatives, generally, the inflammatory conditions are those that involve, at least in part, eicosanoids as pro- or anti-inflammatory signals. Such inflammatory conditions include, for example, fever, allergy, lung disease, respiratory disorders, COVID-19-related cytokine storm, rheumatoid arthritis, lupus, COPD, asthma, ulcerative colitis, Crohn's disease, and glomerulonephritis. In certain aspects, the inflammatory disorders or conditions are those where the afflicted tissue site(s) have arachidonic acid as the dominant PUFA (as compared to other essential fatty acids in the tissue). In other aspects, the inflammatory disorders or conditions are those that cannot be well controlled by aspirin and/or NSAIDs.

In other aspects, the inflammatory condition is characterized by a dysregulation of eicosanoid production as compared to normal subjects. For example, the subject may have dysregulated levels of LOX pathway leukotrienes such as LTA4, LTC4, LTD4, and LTE4. In another example, the subject may have dysregulated levels of COX pathway second messengers, such as prostaglandins, including prostacyclins, and thromboxanes. In another example, the subject may have dysregulated levels of cytochrome P450 pathway products, such as EETs. In other aspects, the subject may suffer from dysregulation of LOX, COX, and/or cytochrome P450 pathway s.

In other aspects, stabilizing, bis-allylic isotopically modified arachidonic acid is administered to a subject having an inflammatory disorder or condition associated with COX, LOX, and cytochrome P450 pathways. The subject having such an inflammatory disorder or condition can be selected by assaying for elevated levels of eicosanoids in the subject, including EETs (Cytochrome P450 pathway), prostanoids including prostaglandins, prostacyclin, and thromboxane (COX pathway), and HETEs, leukotrienes, and lipoxins (LOX pathway). Eicosanoid levels can be determined as known in the art, for example, by eicosanoid ELISA assays with serum from the patient.

Furthermore, stabilizing, bis-allylic isotopically modified arachidonic acid or derivatives thereof is administered to a subject having inflammation-induced elevated procoagulant factors, reducing thrombosis. Platelet coagulation and thromboxanes such as thromboxane A2 (TXA2) can be easily measures in serum or plasma to deduce the aggregation parameters using various methods well known in the art. Coagulation further augments inflammation, causing the vicious cycle. There are many systemic inflammatory diseases characterized by thrombotic tendency, including Behçet's disease (BD), anti-neutrophil cytoplasmic antibody (ANCA) associated vasculitis, Takayasu arteritis, rheumatoid arthritis, systemic lupus erythematosus, antiphospholipid syndrome, familial Mediterranean fever, thromboangiitis obliterans (TAO) and inflammatory bowel diseases (Aksu et al, Inflammation-induced thrombosis: mechanisms, disease associations and management. Curr. Pharm. Des. 2012; 18.1478-1493).

In another embodiment, stabilizing, bis-allylic isotopically modified arachidonic acid or derivatives thereof is administered to subjects with inflammatory diseases to inhibit a non-enzymatic lipid peroxidation process which accompanies inflammation and, in a vicious circle, generates yet more inflammatory response.

In certain aspects, the methods of administering the compositions comprising bis-allylic isotopically modified AA is targeted to particular COVID-19 patient subsets. For example, in one aspect, the target patient subset are patients with robust activation and proliferation of CD4+ T-cells, relative lack of cTfh (Circulating Follicular Helper T Cells), some activation of CD8+ TEMRA (CD45RA)-like cell populations (e.g., CD45RA+CD27−CCR7−CX3CR1+, T-bet+) or hyperactivated CD8+ T-cells, and Tbet+ PB (plasmablast B-cells). (See “Immunotype 1” cell population in Mathew et al., Science, 10.1126/science.abc8511 (2020), which is hereby incorporated-by-reference.) In another aspect, the target patient subset are patients characterized by more traditional effector CD8 T cell subsets, less CD4 T cell activation, and proliferating PB and memory B cells. (Id., see “Immunotype 2,” which is hereby incorporated-by-reference.)

In one aspect, the administered deuterated arachidonic acid has at least one deuterium at a bis-allylic position. In another aspect, the deuterated arachidonic acid has multiple deuterium, each deuterium located at a bis-allylic position. In one aspect, the deuterated arachidonic acid has one or two deuterium at least at the C7 bis-allylic position. In other aspects, the administered composition comprises one or more of the following bis-allylic site-specifically deuterated arachidonic species (see also FIG. 2): 7,7-D2-arachidonic acid and esters thereof, 10,10-D2-arachidonic acid and esters thereof, 13,13-D2-arachidonic acid and esters thereof, 7,7,10,10-D4-arachidonic acid and esters thereof; 7,7,13,13-D4-arachidonic acid and esters thereof; 10,10,13,13-D4-arachidonic acid and esters thereof; 7,7,10,10,13,13-D6-arachidonic acid and esters thereof and combinations of these arachidonic species thereof. While each of these arachidonic acids have two deuterium at each bis-allylic position, they can also have one or two deuterium at each bis-allylic position. In other aspects, these species are tritiated rather than deuterated.

In another aspect, the deuterated arachidonic acid can have additional isotopic modifications, including ¹³C at C7, C10, and/or C13 positions. For example, 7-¹³C-7,7-D2-arachidonic acid and esters thereof, 7-¹³C-10,10-D2-arachidonic acid and esters thereof; 7-¹³C-13,13-D2-arachidonic acid and esters thereof, 7-¹³C-7,7,10,10-D4-arachidonic acid and esters thereof; 7-13 C-7,7,13,13-D4-arachidonic acid and esters thereof; 7-¹³C-10,10,13,13-D4-arachidonic acid and esters thereof, 7-¹³C-7,7,10,10,13,13-D6-arachidonic acid and esters thereof; and combinations of these arachidonic species thereof. In each of these species, C7 is isotopically modified with 13C, and can additionally or alternatively have ¹³C at the C10 and/or C13 positions. Combining deuteration and ¹³C substitution at bis allylic sites can result in larger protective effects, such as for example 7,7,10,10,13,13-D6, 7,10,13-¹³C3-arachidonic acid and esters thereof.

In certain aspects, the administered isotopically modified PUFA(s) is in a pharmaceutically acceptable form, including an ingestible form such as a tablet, capsule, gel, gel-capsule, solution, injectable needle, powder, or liposome. In some embodiments, the modified PUFAs can be in the form (e.g., formulated or derivatized) of a triglyceride, a diglyceride, and/or a monoglyceride comprising a fatty acid, a fatty acid mimetic, and/or a fatty acid pro-drug, or esters thereof. As stated, the modified PUFAs can be stabilized at one or more bis-allylic positions including at least one ¹³C atom and/or at least one deuterium atom and/or at least one tritium atom at a bis-allylic position. In some embodiments, the stabilization comprises at least two deuterium atoms at one or more bis-allylic position. In other embodiments, the stabilization utilizes an amount of isotopes that is above the naturally-occurring abundance level.

For example, regarding hydrogen substitution with deuterium, deuterium has a natural abundance of roughly 0.0156% of all naturally occurring hydrogen in the oceans on earth. Thus, a PUFA having greater than the natural abundance of deuterium may have greater than this level or greater than the natural abundance level of roughly 0.0156% of its hydrogen atoms reinforced with deuterium, such as 0.02%, but preferably greater than 5%, 10%, 10,15%, 20%, 25%, 30%, 35%, 40%, 45%, 50%, 65%, 60%, 65%, 70%, 75%, 80, 85%, 90%, 95%, or 100% of deuterium with respect to one or more hydrogen atoms of the administered isotopically-modified PUFA molecules.

In certain aspects, the administered isotopically-modified composition has an isotopic purity of from about 20%-99%. In other aspects, the administered composition comprising an isotopically modified PUFA or ester thereof is administered to a patient along with nonstabilized fatty acids, fatty acid mimetics, or fatty acid pro-drugs. In some embodiments, the administered isotopically stabilized fatty acids, fatty acid mimetics, or fatty acid pro-drugs comprise between about 1% and 100%, between about 5% and 75%, between about 10% and 30%, or about 20% or more of the total amount of fatty acids, fatty acid mimetics, or fatty acid pro-drugs administered to the patient. In some embodiments, the patient ingests the fatty acid, fatty acid mimetic, or fatty acid pro-drug. In some embodiments, a cell or tissue of the patient maintains a sufficient concentration of the fatty acid, fatty acid mimetic, fatty acid pro-drug, triglyceride, diglyceride, and/or monoglyceride to prevent autooxidation of the naturally occurring polyunsaturated fatty acid, mimetic, or ester prodrug.

Some embodiments further comprise co-administering an antioxidant. In some embodiments, the antioxidant is Coenzyme Q, idebenone, mitoquinone, or mitoquinol. In other embodiments, the antioxidant is a mitochondrially-targeted antioxidant. In some embodiments, the antioxidant is a vitamin, vitamin mimetic, or vitamin pro-drug. In other embodiments, the antioxidant is a vitamin E, vitamin E mimetic, vitamin E pro-drug, vitamin C, vitamin C mimetic, and/or vitamin C pro-drug. In other embodiments, COX, LOX, or dual COX/LOX inhibitors are co-administered, including but not limited to COX1 inhibitors, COX 2 inhibitors, 5-LOX inhibitors, and dual COX-2/5-LOX inhibitors, as known in the art.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the present disclosure are set forth with particularity in the appended claims. A better understanding of the features and advantages of the present disclosure will be obtained by reference to the following detailed description that sets forth illustrative embodiments, in which the principles of the present disclosure are utilized, and the accompanying drawings (also “Figure” and “FIG.” herein), of which:

FIG. 1. Different bis-allylic sites may play a different role in oxidation of arachidonic acid.

FIG. 2. Bis-allylic site-specific isotopically modified arachidonic acids. The bis-allylic positions are located at the C7, C10, and C13 positions. “D” represents deuterium.

FIGS. 3-6. Groups of female (f) and male (m) mice received a 6-week course of dietary (H) AA or a 6-week course of deuterated arachidonic acid (D6-ARA, 15 mg/day) followed by single intranasal administration of LPS. Control mice (norm) did not receive the single intranasal administration of LPS. Mice that received dietary AA showed an increase of the alveolar lumen area (FIG. 3 is the median; FIG. 4 is the average) and an increase of the interalveolar septa thickness (FIG. 5 is the median; FIG. 6 is the average), which is likely associated with stronger edema and inflammatory infiltration. Mice that received D6-ARA exhibited alveolar lumen area and thickness consistent with controls. In general, the data of FIGS. 3-6 indicate a lesser degree of inflammatory lesion of the lungs after course of D-form compared to H-form. Preliminary data (not shown) indicates that D6-ARA had the best protective effect as compared to other deuterated PUFAs.

### DETAILED DESCRIPTION

In the following detailed description, reference is made to the accompanying figures, which form a part hereof. In the figures, similar symbols typically identify similar components, unless context dictates otherwise. The illustrative embodiments described in the detailed description, figures, and claims are not meant to be limiting. Other embodiments may be utilized, and other changes may be made, without departing from the scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Although certain embodiments and examples are disclosed below, inventive subject matter extends beyond the specifically disclosed embodiments to other alternative embodiments and/or uses, and to modifications and equivalents thereof. Thus, the scope of the claims appended hereto is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments, however, the order of description should not be construed to imply that these operations are order dependent. Additionally, the structures, systems, and/or devices described herein may be embodied as integrated components or as separate components.

For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

### Inflammation-Related Disorders

Inflammation-related disorders represent a heterogeneous group of diseases. For example, inflammation-related disorders include, but are not limited to, asthma, rheumatoid arthritis, juvenile chronic arthritis, osteoarthritis, myositis, Crohn's disease, gastritis, colitis, ulcerative colitis, inflammatory bowel disease, proctitis, pelvic inflammatory disease, systemic lupus erythematosus, rhinitis, conjunctivitis, scleritis, chronic inflammatory polyneuropathy, Lyme disease, psoriasis, dermatitis, eczema, autoimmune disorders, and atherosclerosis.

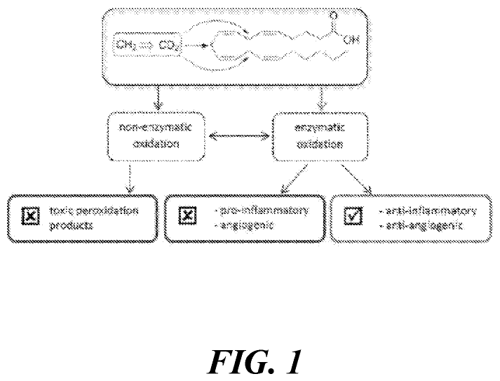

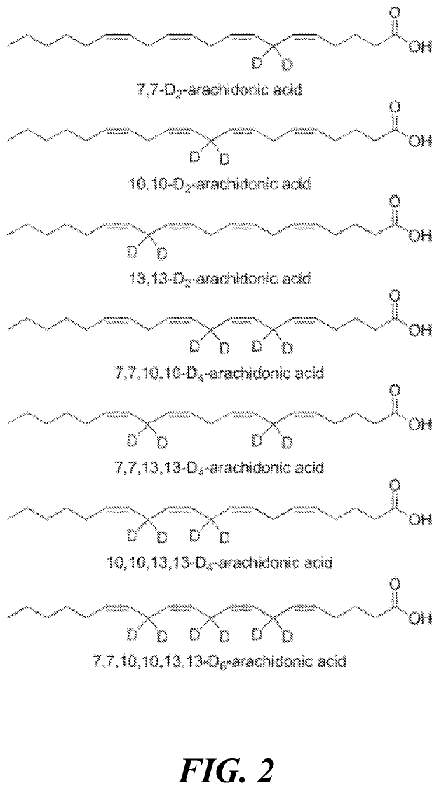

Inflammation-related disorders often involve increased generation of ROS (reactive oxygen species) and lipid processing abnormalities (Winyard P G et al., Free radicals and inflammation. Burkhauser, Basel, 2000; Filippo et al., Journal of Alzheimer's Disease (2010); 20, S369-S379). The combination of ROS and lipid processing abnormalities leads to increased PUFA peroxidation that generates toxic reactive carbonyl compounds which inflict further damage at numerous other sites within the cell (Negre-Salvayre A et al Br J Pharmacol 2008; 153:6-20). Consequently, the detection of oxidatively damaged biomolecules and the presence of ROS in inflammatory states both implicate elevated oxidative stress levels. As shown in FIG. 1, Different bis-allylic sites may play a different role in oxidation of arachidonic acid.

Lipid peroxidation is important in atherosclerosis and in worsening of initial tissue injury caused by ischemic or traumatic brain damage. Atherosclerosis is a lipid storage disorder associated with inflammatory response (Ross R, New Engl J Med. 1999; 340:115-127). Oxidation of PUFA-rich low-density lipoprotein (LDL) was suggested to be a major risk factor for atherosclerosis and for hypertension associated with atherosclerosis. Angiotensin II (Ang-II) is involved in this process, enhancing macrophage lipid peroxidation both in vivo and in vitro (Keidar S. Life Sci. 1998; 63:1-11). Despite the importance of oxidative stress in the etiology of atherosclerosis, the success of antioxidant therapies aiming to reduce lipid peroxidation has so far been limited (Stocker R. et al., Physiol Rev 2004; 84:1381-1478).

Rheumatoid arthritis may be associated with inflammation and PUFA peroxidation through a reperfusion injury mechanism. The synovial cavity normally has a negative pressure. When the joint is exercised, vascular patency is maintained, allowing for nutrition of the avascular cartilage. In rheumatoid synovitis, the cavity pressure is raised and upon movement this pressure exceeds the capillary perfusion pressure, causing collapse of the blood vessels. This leads to the production of multiple episodes of ‘hypoxic-reperfusion injury’ generating ROS which oxidize several targets, including, but not limited to: IgG, inducing rheumatoid factor production; Hyaluronan, leading to hyaluronan fragmentation products which may alter immune function; PUFAs, generating reactive carbonyls which alter T cell/macrophage interactions; and lipoproteins, leading to the production of monocyte chemotactic peptides. Progressive hypoxia alters immune function, predominantly by calcium mediated pathways. (Mapp P I et al., Br Med Bull 1995; 51:419-436). Various PUFA peroxidation products including, but not limited to, conjugated dienes, isoprostanes and reactive aldehydes such as HNE are detectable in plasma and synovial fluid of rheumatoid arthritis and osteoarthritis patients (Selley M L Annals Rheum Disease 1992; 51:481-484).

As used herein “inflammation-related disorders” encompasses conditions that are not themselves considered to be inflammatory conditions. For example, as stated previously, platelet coagulation and thromboxanes can augment inflammation, vice-versa, causing a vicious cycle. In addition, the present disclosure encompasses systemic inflammatory diseases characterized by thrombotic tendencies, including Behçet's disease (BD), anti-neutrophil cytoplasmic antibody (ANCA) associated Vasculitis, Takayasu arteritis, rheumatoid arthritis, systemic lupus erythematosus, antiphospholipid syndrome, familial Mediterranean fever, thromboangiitis obliterans (TAO) and inflammatory bowel diseases (Aksu et al, Inflammation-induced thrombosis: mechanisms, disease associations and management. Curr. Pharm. Des. 2012; 18:1478-1493). Similarly, “inflammation-related disorders” include inflammation that can arise, at least in part, from the non-enzymatic chain reaction of PUFA damage, known as lipid peroxidation (LPO), which may be initiated by enzymes or by small molecule ROS. This pro-inflammatory non-enzymatic component is impervious to antioxidant approaches due to the structural aspects of the lipid bilayers making up membranes Inflammation associated LPO can be initiated through various mechanisms. (Zhang et al, Myeloperoxidase Functions as a Major Enzymatic Catalyst for Initiation of Lipid Peroxidation at Sites of Inflammation, J Blot Chem 2002 doi: 10.1074jbc.M209124200).

Oxidative stress at sites with chronic inflammation can also cause genetic changes. Reactive carbonyl products of PUFA peroxidation can react with DNA bases and change their complementarity pattern (Esterbauer H et al., Free Rad Biol Med 1991; 11:81-128). The development of mutations in the p53 tumor suppressor gene and other key regulatory genes can convert inflammation into chronic disease in rheumatoid arthritis and other inflammatory disorders (Tak P P et al., Immunology Today 2000; 21:78-82).

Arachidonic acid (AA), an essential polyunsaturated fatty acid (PUFA) present in the human diet, is a vital building block for biological membranes. Besides this function, AA is a substrate of several enzymes (COX1, COX2, various LOX enzymes, etc.), yielding multiple pro-inflammatory and pro-blood clotting eicosanoid mediators such as prostaglandins, leukotrienes and thromboxanes, which then initiate further inflammatory cascades. Inflammation increases the level of reactive oxygen species (ROS) which in turn initiate non-enzymatic chain reaction of AA peroxidation in lipid membranes (LPO), leading to multiple toxic products. Both enzymatic and non-enzymatic AA oxidative transformations happen at any of the three bis-allylic sites within the AA molecule. The key rate limiting step for both types of transformation, termed hydrogen abstraction (a C—H bond cleavage), defines the rate of the whole process. Thus, the present disclosure provides therapeutic methods based on the concept that AA oxidation plays a pivotal role in multiple aspects of inflammatory disorders, including cytokine storm and thrombotic complications.

COX and LOX inhibitors can be employed to down-regulate this process, with various efficacy. In contrast, the methods of the present disclosure rely on deuteration of the three oxidation prone sites within PUFA molecules, to substantially slow down the rate limiting step of oxidation (both enzymatic and non-enzymatic) via the isotope effect (IE). Made in high yield via a catalytic process, such D-PUFAs were tested in a lipopolysaccharide (LPS)-inflicted lung inflammation model in mice, a well-known lung inflammation model. (See Example 1.)

Intranasal administration of lipopolysaccharide (LPS), a bacterial endotoxin, has been shown to initiate a pro-inflammatory response in the lungs of mice. For example, the mouse LPS model has been used as a model for human acute lung injury (ALI), as the mouse lung parenchyma is damaged by the generation and release of proteases and reactive oxygen and nitrogen species produced by activated lung macrophages and transmigrated neutrophils in the interstitial and alveolar compartments. The end results are microvascular injury and diffuse alveolar damage with intrapulmonary hemorrhage, edema, and fibrin deposition (Johnson and Ward, J. Clin. Invest., (1974), 54:349-357; Flierl et al., Med. Hypothesis Res., (2006), 3:727,738), which are also features in patients with ALI and the acute respiratory distress syndrome (ARDS) (Kabir et al., Shock, (2002), 17:300-303; Ward, Am. J. Pathol., (1996), 149:1081-1086). This LPS model recapitulates aspects of the inflammatory cascades that are associated with pulmonary inflammation associated with lung disease in humans, and so is useful as a screen for compounds that may disrupt these cascades and attenuate or abort the disease process. As discussed herein, in some embodiments, the D-PUFAs of the present disclosure are administered to break the inflammatory cascade of specific cytokines and chemokines known to induce and amplify lung inflammation. Thus, for example, one can experimentally assess the efficacy of the D-PUFAs by examining whether the levels of inflammation associated markers are reduced. Two such markers, monocyte chemoattractant protein (MCP-1), and tumor necrosis factor alpha (TNF-a), are found to be associated with early and chronic infection and inflammation in humans and are mirrored in rodent models of airway inflammation including the mouse LPS pulmonary inflammation model.

In some embodiments, the D-PUFAs of the present disclosure are administered to a subject having an inflammation-related disorder as described herein, including inflammation-related disorders associated with increased levels in ROS and/or lipid peroxidation in cells. Inflammation-related disorders include, but are not limited to, asthma, rheumatoid arthritis, juvenile chronic arthritis, osteoarthritis, myositis, Crohn's disease, gastritis, colitis, ulcerative colitis, inflammatory bowel disease, proctitis, pelvic inflammatory disease, systemic lupus erythematosus, rhinitis, conjunctivitis, scleritis, chronic inflammatory polyneuropathy, Lyme disease, psoriasis, dermatitis, eczema, autoimmune disorders, atherosclerosis, and COVID-19.

For aspects that comprise the administration of bis-allylic modified arachidonic acid and its derivatives, generally, the inflammatory conditions are those that involve, at least in part, eicosanoids as pro- or anti-inflammatory signals. Such inflammatory conditions include, for example, fever, allergy, lung disease, respiratory disorders, COVID-19-related cytokine storm, rheumatoid arthritis, lupus, COPD, asthma, ulcerative colitis, Crohn's disease, and glomerulonephritis. In certain aspects, the inflammatory disorders or conditions are those where the afflicted tissue site(s) have arachidonic acid as the dominant PUFA (as compared to other essential fatty acids in the tissue). In other aspects, the inflammatory disorders or conditions are those that cannot be well controlled by aspirin and/or NSAIDs.

In some embodiments, the D-PUFAs of the present disclosure are administered to particular COVID-19 patient subpopulations characterized by immune cell profiles. In one embodiment, the D-PUFAs of the present disclosure are administered to a COVID-19 patient subpopulation characterized by the “Immunotype 1” immune cell profile, i.e., activated and proliferating CD4+ T-cells, relative lack of cTfh, proliferating effector/exhausted CD8+ T-cells, and T-bet+PB involvement. (Mathew et al., 10.1126/science.abc8511, Science, (2020).) In another embodiment, the D-PUFAs of the present disclosure are administered to a COVID-19 patient subpopulation characterized by the “Immunotype 2” immune cell profile, i.e., more traditional CD8+ T-cell subsets, less CD4 T-cell activation, and proliferating PB and memory B-cells. (Id.)

Different bis-allylic sites may play a different role in oxidation of arachidonic acid. For example, in one embodiment, the anti-inflammatory methods require at least LOX inhibition by administering AA deuterated at least at C7 in AA. In the case of COVID-19, the primary MOA (mechanism of action) is inhibition of 5-LOX-mediated LTB4 biosynthesis after lectin-pathway-mediated release of AA by Ca2+-dependent PLA2, which then prevents release of IL-6, a major cause for cytokine storm.

Some aspects of the present disclosure arise from: (1) an understanding that while essential PUFAs are vital for proper functioning of lipid membranes, and in particular of the mitochondrial membranes, their inherent drawback, i.e., the propensity to be oxidized by ROS with detrimental outcome, is implicated in inflammatory disorders; (2) antioxidants cannot prevent PUFA peroxidation due to stochastic nature of the process and the stability of PUFA peroxidation products (reactive carbonyls) to antioxidant treatment, and (3) the ROS-driven damage of oxidation-prone sites within PUFAs may be overcome by using an approach that makes them less amenable to such oxidations, without compromising any of their beneficial physical properties. Some aspects of the present disclosure describe the use of the isotope effect to achieve this, only at sites in essential PUFAs and PUFA precursors that matter most for oxidation, while other aspects contemplate other sites in addition to those that matter most for oxidation.

Isotopically labeled embodiments should have minimal or non-existent effects on important biological processes. For example, the natural abundance of isotopes present in biological substrates implies that low levels of isotopically labeled compounds should have negligible effects on biological processes. Additionally, hydrogen atoms are incorporated into biological substrates from water, and is it known that the consumption of D20, or heavy water, does not pose a health threat to humans. See, e.g., “Physiological effect of heavy water.” Elements and isotopes: formation, transformation, distribution. Dordrecht: Kluwer Acad. Publ. (2003) pp. 111-112 (indicating that a 70 kg person might drink 4.8 liters of heavy water without serious consequences). Moreover, many isotopically labeled compounds are approved by the U.S. Food & Drug Administration for diagnostic and treatment purposes.

### D-PUFA Composition for Administration

In some embodiments, an isotopically modified polyunsaturated fatty acid or a mimetic refers to a compound having structural similarity to a naturally occurring PUFA that is stabilized chemically or by reinforcement with one or more isotopes, for example ¹³C and/or deuterium or tritium. Generally, if deuterium or tritium is used for reinforcement, one or both hydrogens on a methylene group may be reinforced.

Some aspects of the present disclosure provide compounds that are analogues of essential PUFAs with one, several, or all bis-allylic positions substituted with heavy isotopes. In some embodiments, the CH₂ groups, which will become the bis-allylic position in a PUFA upon enzymatic conversion, are substituted with one or two heavy isotopes. Such compounds are useful for the prevention or treatment of diseases in which PUFA oxidation is a factor or can contribute to disease progression.

The bis-allylic position generally refers to the position of the polyunsaturated fatty acid or mimetic thereof that corresponds to the methylene groups of 1,4-diene systems. The pro-bis-allylic position refers to the methylene group that becomes the bis-allylic position upon enzymatic desaturation.

In some embodiments, the chemical identity of PUFAs, i.e., the chemical structure without regard to the isotope substitutions or substitutions that mimic isotope substitutions, remains the same upon ingestion. For instance, the chemical identity of essential PUFAs, that is, PUFAs that mammals such as humans do not generally synthesize, may remain identical upon ingestion. In some cases, however, PUFAs may be further extended/desaturated in mammals, thus changing their chemical identity upon ingestion. Similarly with mimetics, the chemical identity may remain unchanged or may be subject to similar extension/desaturation. In some embodiments, PUFAs that are extended, and optionally desaturated, upon ingestion and further metabolism may be referred to as higher homologs.

In some embodiments, naturally-occurring abundance level refers to the level of isotopes, for example ¹³C and/or deuterium that may be incorporated into PUFAs that would be relative to the natural abundance of the isotope in nature. For example, ¹³C has a natural abundance of roughly 1% ¹³C atoms in total carbon atoms. Thus, the relative percentage of carbon having greater than the natural abundance of ¹³C in PUFAs may have greater than the natural abundance level of roughly 1% of its total carbon atoms reinforced with ¹³C, such as 2%, but preferably about 5%, 10%, %15%, 20%, 25%, 30%, 35%, 40%, 45%,50%, 65%, 60%, 65%, 70%, 75%, 80%, 85%, 90%, 95%, or 100% of ¹³C with respect to one or more carbon atoms in each PUFA molecule. In other embodiments, the percentage of total carbon atoms reinforced with ¹³C is at least 5%, 10%, 15%, 20%, 25%, 30%, 35%, 40%, 45%,50%, 65%, 60%, 65%, 70%, 75%, 80%, 85%, 90%, 95%, or 100%.

Regarding hydrogen, in some embodiments, deuterium has a natural abundance of roughly 0.0156% of all naturally occurring hydrogen in the oceans on earth. Thus, a PUFA having greater that the natural abundance of deuterium may have greater than this level or greater than the natural abundance level of roughly 0.0156% of its hydrogen atoms reinforced with deuterium, such as 0.02%, but preferably about 5%, 10%, 15%, 20%, 25%, 30%, 35%, 40%, 45%, 50%, 65%, 60%, 65%, 70%, 75%, 80%, 85%, 90%, 95%, or 100% of deuterium with respect to one or more hydrogen atoms in each PUFA molecule. In other embodiments, the percentage of total hydrogen atoms reinforced with deuterium is at least 5%, 10%, 15%, 20%, 25%, 30%, 35%, 40%, 45%, 50%, 65%, 60%, 65%, 70%, 75%, 80, 85%, 90%, 95% or 100%.

In some aspects, a composition of PUFAs contains both isotopically modified PUFAs and isotopically unmodified PUFAs. The isotopic purity is a comparison between a) the relative number of molecules of isotopically modified PUFAs, and b) the total molecules of both isotopically modified PUFAs and PUFAs with no heavy atoms. In some embodiments, the isotopic purity refers to PUFAs that are otherwise the same except for the heavy atoms.

In some embodiments, isotopic purity refers to the percentage of molecules of an isotopically modified PUFAs in the composition relative to the total number of molecules of the isotopically modified PUFAs plus PUFAs with no heavy atoms. For example, the isotopic purity may be about 5%, %10%,15%, 20%,25%, 30%,35%, 40%,45%,50%,65%, 60%, 65%, 70%, 75%, 80%, 85%, 90%, 95%, or 100% of the molecules of isotopically modified PUFAs relative to the total number of molecules of both the isotopically modified PUFAs plus PUFAs with no heavy atoms. In other embodiments, the isotopic purity is at least 5%, 10%, 15%, 20%, 25%, 30%, 35%, 40%, 4%5% 50%, 65%, 60%, 65%, 70%, 75%, 80, 85%, 90%, 95%, or 100%. In some embodiments, isotopic purity of the PUFAs may be from about 10%-100%, 10%-95%, 10%-90%, 10%-85%, 10%-80%, 10%-75%, 10%-70%, 10%-65%, 10%-60%, 10%-55%, 10%, 50%, 10%-45%, 10%-40%, 10%-35%, 10%-30%, 10%-25%, or 10%-20% of the total number of molecules of the PUFAs in the composition. In other embodiments, isotopic purity of the PUFAs may be from about 15%-100%, 15%-95%, 15%-90%, 15%-85%, 15%-80%, 15%-75%, 15%, 70%, 15%-65%, 15%-60%, 15%-55%, 15%-50%, 15%-45%, 15%-40%, 15%-35%, 15%-30%, 15%-25%, or 15%-20% of the total number of molecules of the PUFAs in the composition. In some embodiments, isotopic purity of the PUFAs may be from about 20%-100%, 20%-95%, 20%-90%, 20%-85%, 20%-80%, 20%-75%, 20%-70%, 20%-65%, 20%-60%, 20%-55%, 20%,50%, 20%-45%, 20%-40%, 20%-35%, 20%-30%, or 20%-25% of the total number of molecules of the PUFAs in the composition. Two molecules of an isotopically modified PUFA out of a total of 100 total molecules of isotopically modified PUFAs plus PUFAs with no heavy atoms will have 2% isotopic purity, regardless of the number of heavy atoms the two isotopically modified molecules contain

In some aspects, an isotopically modified PUFA molecule may contain one deuterium atom, such as when one of the two hydrogens in a methylene group is replaced by deuterium, and thus may be referred to as a “D1” PUFA. Similarly, an isotopically modified PUFA molecule may contain two deuterium atoms, such as when the two hydrogens in a methylene group are both replaced by deuterium, and thus may be referred to as a “D2” PUFA. Similarly, an isotopically modified PUFA molecule may contain three deuterium atoms and may be referred to as a “D3” PUFA. Similarly, an isotopically modified PUFA molecule may contain four deuterium atoms and may be referred to as a “D4” PUFA. In some embodiments, an isotopically modified PUFA molecule may contain five deuterium atoms or six deuterium atoms and may be referred to as a “D5” or “D6” PUFA, respectively.

The number of heavy atoms in a molecule, or the isotopic load, may vary. For example, a molecule with a relatively low isotopic load may contain about 1, 2, 3, 4, 5, or 6 deuterium atoms. A molecule with a moderate isotopic load may contain about 10, 11, 12, 13, 14, 15, 16, 17, 18, 19, or 20 deuterium atoms. In a molecule with a very high load, every hydrogen may be replaced with a deuterium (or tritium). Thus, the isotopic load refers to the percentage of heavy atoms in each PUFA molecule. For example, the isotopic load may be about 5%, 10%, 15%, 20%, 25%, 30%, 35%, 40%, 45%, 50%, 65%, 60%, 65%, 70%, 75%, 80%, 85%, 90%, 95%, or 100% of the number of the same type of atoms in comparison to a PUFA with no heavy atoms of the same type (e.g. hydrogen would be the “same type” as deuterium or tritium, and carbon would be the “same type” as ¹³C). In some embodiments, the isotopic load is at least 5%, 10%, 15%, 20%, 25%, 30%, 35%, 40%, 45%, 50%, 65%, 60%, 65%, 70%, 75%, 80%, 85%, 90%, 95%, or 100%.

Unintended side effects are expected to be reduced where there is high isotopic purity in a PUFA composition but low isotopic load in a given molecule. For example, the metabolic pathways will likely be less affected by using a PUFA composition with high isotopic purity but low isotopic load.

One will readily appreciate that when one of the two hydrogens of a methylene group is replaced with a deuterium atom, the resultant compound may possess a stereocenter. In some embodiments, it may be desirable to use racemic compounds. In other embodiments, it may be desirable to use enantiomerically pure compounds. In additional embodiments, it may be desirable to use diastereomerically pure compounds. In some embodiments, it may be desirable to use mixtures of compounds having enantiomeric excesses and/or diastereomeric excesses of about 5%, 10%, 15%, 20%, 25%, 30%, 350% 40%, 45%, 50%, 65%, 60%, 65%, 70%, 75%, 80%, 85%, 90%, 95%, or 100%. In other embodiments, the enantiomeric excesses and/or diastereomeric excesses is at least 5%, 10%, 15%, 20%, 25%, 30%, 35%, 40%, 45%, 50%, 65%, 60%, 65%, 70%, 75%, 80%, 85%, 90%, 95%, or 100%. In some embodiments, it may be preferable to utilize stereochemically pure enantiomers and/or diastereomers of embodiments such as when contact with chiral molecules is being targeted for attenuating oxidative damage. However, in many circumstances, non-chiral molecules are being targeted for attenuating oxidative damage. In such circumstances, embodiments may be utilized without concern for their stereochemical purity. Moreover, in some embodiments, mixtures of enantiomers and diastereomers may be used even when the compounds are targeting chiral molecules for attenuating oxidative damage. In some embodiments, the administered compositions comprise a bis-allylic isotopically modified essential PUFAs, or derivatives thereof, that are substantially in the cis isomeric form. For example, the administered composition can comprise bis-allylic isotopically modified essential PUFAs that are at least 90%, 91%, 92%, 93%, 94%, 95%, 96%, 97%, 98%, 99%, or 100% in the cis isomeric form (and the remaining in the trans isomeric form).

In some aspects, isotopically modified PUFAs impart an amount of heavy atoms in a particular tissue. Thus, in some aspects, the amount of heavy molecules will be a particular percentage of the same type of molecules in a tissue. For example, the number of heavy molecules may be about 1%-100% of the total amount of the same type of molecules. In some aspects, 10-50% the molecules are substituted with the same type of heavy molecules. In some embodiments, a compound with the same chemical bonding structure as an essential PUFA but with a different isotopic composition at particular positions will have significantly and usefully different chemical properties from the unsubstituted compound. The particular positions with respect to oxidation, including oxidation by ROS, comprise bis-allylic positions of essential polyunsaturated fatty acids and their derivatives. The essential PUFAs isotopically reinforced at bis-allylic positions will be more stable to the oxidation.

Accordingly, some aspects of the present disclosure provide for particular methods of using compounds of Formula (1) or salts thereof, wherein the bis-allylic sites can be further reinforced with carbon-13.

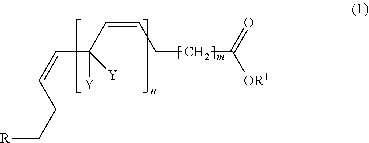

In Formula (1) above, n=1-5; m=1-10; R=H, C₃H; R¹—=H, alkyl, or a cation; Y=hydrogen, deuterium, or tritium so long as there is at least one Y that is substituted with a deuterium or tritium; and wherein each bis-allylic carbon (i.e., the carbon that is covalently bonded to the Ys) is either ¹³C or C (i.e., ¹²C, not isotopically modified/substituted). All other carbon-hydrogen bonds in the PUFA molecule, including carbon-hydrogen bonds at a pro-bis-allylic position, may optionally contain deuterium or tritium and/or carbon-13. Compositions comprising the compounds of Formula (1) have an amount of substituted heavy atoms that is above the naturally occurring abundance level of the corresponding heavy atoms.

Exemplary species of Formula (1) include, but are not limited to the following omega-3 and omega-6 essential PUFAs (and salts and esters thereof): 11-D-linoleic acid; 11,11-D2-linoleic acid; 11-D-linolenic acid; 11,11-D2-linolenic acid; 14-D-linolenic acid; 14,14-D2-linolenic acid; 11,14-D2-linolenic acid; 11,11,14,14-D4-linolenic acid; 7,7-D2-arachidonic acid; 10,10-D2-arachidonic acid; 13,13-D2-arachidonic acid; 7,7,10,10-D4-arachidonic acid; 7,7,13,13-D4-arachidonic acid; 10,10,13,13-D4-arachidonic acid; 7,7,10,10,13,13-D6-arachidonic acid; 7,7-D2-eicosapentaenoic acid; 10,10-D2-eicosapentaenoic acid; 13,13-D2-eicosapentaenoic acid; 16,16-D2-eicosapentaenoic acid; 7,7,10,10-D4-eicosapentaenoic acid; 7,7,13,13-D4-eicosapentaenoic acid; 7,7,16,16-D4-eicosapentaenoic acid; 10,10,13-D4-eicosapentaenoic acid; 10,10,16,16-D4-eicosapentaenoic acid; 13,13,16,16-D4-eicosapentaenoic acid; 7,7,10,10,13,13-D6-eicosapentaenoic acid; 7,7,10,10,16,16-D6-eicosapentaenoic acid; 7,7,13,13,16,16-D6-eicosapentaenoic acid; 10,10,13,13,16,16-D6-eicosapentaenoic acid; 7,7,10,10,13,13,16,16-D8-eicosapentaenoic acid; 6,6-D2-docosahexaenoic acid; 9,9-D2-docosahexaenoic acid; 12,12-D2-docosahexaenoic acid; 15,15-D2-docosahexaenoic acid; 18,18-D2-docosahexaenoic acid; 6,6,9,9-D4-docosahexaenoic acid; 6,6,12,12-D4-docosahexaenoic acid; 6,6,15,15-D4-docosahexaenoic acid; 6,6,18,18-D4-docosahexaenoic acid; 9,9,12,12-D4-docosahexaenoic acid; 9,9,15,15-D4-docosahexaenoic acid; 9,9,18,18-D4-docosahexaenoic acid; 12,12,15,15-D4-docosahexaenoic acid; 12,12,18,18-D4-docosahexaenoic acid; 15,15,18,18-D4-docosahexaenoic acid; 6,6,9,9,12,12-D6-docosahexaenoic acid; 6,6,9,9,15,15-D6-docosahexaenoic acid; 6,6,9,9,18,18-D6-docosahexaenoic acid; 9,9,12,12,15,15-D6-docosahexaenoic acid; 9,9,12,12,18,18-D6-docosahexaenoic acid; 12,12,15,15,18,18-D6-docosahexaenoic acid; 6,6,9,9,12,12,15,15-D8-docosahexaenoic acid; 6,6,9,9,12,12,18,18-D8-docosahexaenoic acid; 6,6,9,9,15,15,18,18-D8-docosahexaenoic acid; 6,6,15,15,18,18-D6-docosahexaenoic acid; 9,9,15,15,18,18-D6-docosahexaenoic acid; 6,6, 12,12,15,15-D6-docosahexaenoic acid; 6,6,12,12,18,18-D6-docosahexaenoic acid; 6,6,12,12,15,15,18,18-D8-docosahexaenoic acid; 9,9,12,12,15,15,18,18-D8-docosahexaenoic acid; 6,6,9,9,12,12,15,15,18,18-D10-docosahexaenoic acid. For the above-listed species, the POSA understands the numbering convention, where numbers preceding “D” indicate the carbon position and the number following “D” indicates the total number of deuterium. Each of the positions in the above-listed species are the carbon positions of bis-allylic sites. With each of these non-limiting examples, the modified fatty acid can be in a derivative form, including an ester form. Further, with each of these non-limiting examples, the fatty acid can be further modified by isotopically modifying one or more bis-allylic carbons with ¹³C. Further, with each of these non-limiting examples, the fatty acid can be substituted with tritium instead of deuterium at the specified locations. Yet further, with each of these non-limiting examples, the fatty acid can be further modified at one or more pro-bis-allylic positions with ¹³C and/or deuterium or tritium. In related aspects, the administered compositions can comprise a mixture of two or more of the above-listed species.

Essential PUFAs are biochemically converted into higher homologues by desaturation and elongation. Therefore, some sites which are not bis-allylic in the precursor PUFAs will become bis-allylic upon biochemical transformation. Such sites then become sensitive to oxidation, including oxidation by ROS. In a further embodiment, such pro-bis-allylic sites, in addition to existing bis-allylic sites are reinforced by isotope substitution as shown below. Accordingly, this aspect of the present disclosure provides for the use of compounds of Formula (2) or salt thereof, where at each bis-allylic position, and at each pro-bis-allylic position, one or more of X or Y atoms may be hydrogen, deuterium, or tritium atoms; so long as at least one bis-allylic or pro-bis-allylic hydrogen is substituted with deuterium or tritium. Further for Formula (2), R¹=alkyl, cation, or H; m=0-10; n=1-5; p=0-10, and when m is 0 then p is at least 1. Further, one or more carbons at bis-allylic or pro-bis-allylic positions may be substituted with ¹³C

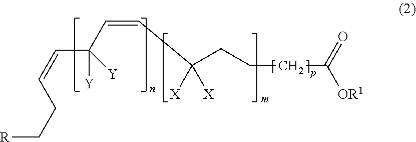

Compositions comprising the compounds of Formula (2) have an amount of substituted heavy atoms that is above the naturally occurring abundance level of the corresponding heavy atoms. In some embodiments, the present disclosure provides for the use of compounds of Formula (3), or salts thereof, differentially reinforced with heavy stable isotopes at selected bis-allylic or pro-bis-allylic positions, to control the relative yield of oxidation at different sites, as shown below, such that any of the pairs of Y¹-Yⁿ and/or X¹-X^{m} at the bis-allylic or pro-bis-allylic positions of PUFAs may contain deuterium atoms. R¹=alkyl, cation, or H; m=1-10; n=1-6; p=1-10; R=H, C₃H; Y=H, D, or T; X=H, D, or T, whereat least one Y or T is substituted with D or T.

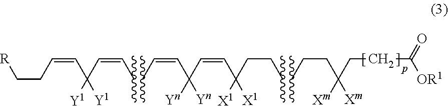

Further, the carbon at bis-allylic or pro-bis-allylic positions can be substituted with ¹³C. Compositions comprising the compounds of Formula (3) have an amount of substituted heavy atoms that is above the naturally occurring abundance level of the corresponding heavy atoms.

Site-specific isotopic modification at bis-allylic and pro-bis-allylic positions is known in the art. As referred to previously, U.S. Pat. No. 10,577,304 provides synthetic schemes for site-specific isotopic modification at bis-allylic and pro-bis-allylic positions for at least linoleic acid, linolenic acid, arachidonic acid, eicosapentaenoic acid (EPA), docosahexaenoic acid (DHA), and derivatives thereof, including esters; the teachings of which are hereby incorporated-by-reference. Such synthetic schemes are also able to provide bis-allylic site-specifically modified essential PUFAs in substantially pure cis isomeric form.

Arachidonic acid is the dominating PUFA in lung tissue with a relative ratio of AA vs DHA vs LA (linolenic acid) of about 60:10:30. In comparison to some other tissues, the relative ratio of AA vs DHA vs. LA in the outer retina is about 10:80:1, and in the cerebral cortex it is about 40:60:1. Thus, in one embodiment, the present methods for treating, preventing, or reducing inflammation of tissues where the dominating PUFA is arachidonic acid. In other embodiments, the present methods for treating, preventing or reducing inflammation comprises administering compositions with a mixture of PUFAs corresponding to the major PUFA species in the target tissue. For example, for the lung, the method can comprise administering D-ARA, D-DHA, and D-LA.

In some embodiments, the inflammatory condition is characterized by a dysregulation of eicosanoid production as compared to normal subjects. For example, the subject may have dysregulated levels of LOX pathway leukotrienes such as LTA4, LTC4, LTD4, and LTE4. In another example, the subject may have dysregulated levels of COX pathway second messengers, such as prostaglandins, including prostacyclins, and thromboxanes. In another example, the subject may have dysregulated levels of cytochrome P450 pathway products, such as EETs. In other aspects, the subject may suffer from dysregulation of LOX, COX, and/or cytochrome P450 pathways.

Thus, in some embodiments, stabilizing, bis-allylic isotopically modified arachidonic acid is administered to a subject having an inflammatory disorder or condition associated with COX, LOX, and cytochrome P450 pathways. The subject having such an inflammatory disorder or condition can be selected by assaying for elevated levels of eicosanoids in the subject, including EETs (Cytochrome P450 pathway), prostanoids including prostaglandins, prostacyclin, and thromboxane (COX pathway), and HETEs, leukotrienes, and lipoxins (LOX pathway). Eicosanoid levels can be determined as known in the art, for example, by eicosanoid ELISA assays with serum from the patient. Leukotrienes.

In other embodiments, the methods of administering the compositions comprising bis-allylic isotopically modified AA is targeted to particular COVID-19 patient subsets. For example, in one aspect, the target patient subset are patients with robust activation and proliferation of CD4+ T-cells, relative lack of cTfh (Circulating Follicular Helper T Cells), some activation of CD8+ TEMRA (CD45RA)-like cell populations (e.g., CD45RA+CD27−CCR7−CX3 CR1+, T-bet+) or hyperactivated CD8+ T-cells, and Tbet+ PB (plasmablast B-cells). (See “Immunotype 1” cell population in Mathew et al., Science, 10. 1126/science.abc8511 (2020), which is hereby incorporated-by-reference.) In another aspect, the target patient subset are patients characterized by more traditional effector CD8 T cell subsets, less CD4 T cell activation, and proliferating PB and memory B cells. (Id., see “Immunotype 2,” which is hereby incorporated by reference.)

In some embodiments, the administered deuterated arachidonic acid has at least one deuterium at a bis-allylic position. In another embodiment, the deuterated arachidonic acid has multiple deuterium, each deuterium located at a bis-allylic position. In one embodiment, the deuterated arachidonic acid has one or two deuterium at least at the C7 bis-allylic position. In other embodiments, the administered composition comprises one or more of the following bis-allylic site-specifically deuterated arachidonic species: 7,7-D2-arachidonic acid and esters thereof; 10,10-D2-arachidonic acid and esters thereof; 13,13-D2-arachidonic acid and esters thereof 7,7,10,10-D4-arachidonic acid and esters thereof 7,7,13,13-D4-arachidonic acid and esters thereof; 10,10,13,13-D4-arachidonic acid and esters thereof; 7,7,10,10,13,13-D6-arachidonic acid and esters thereof, and combinations of these arachidonic species thereof. While each of these arachidonic acids have two deuterium at each bis-allylic position, they can also have one or two deuterium at each bis-allylic position. In other embodiments, these species are tritiated rather than deuterated. Further, the deuterated arachidonic acid can have additional isotopic modifications, including ¹³C at C7, C10, and/or C13 positions. For example, 7-¹³C-7,7-D2-arachidonic acid and esters thereof; 7-¹³C-10,10-D2-arachidonic acid and esters thereof, 7-¹³C-13,13-D2-arachidonic acid and esters thereof, 7-¹³C-7,7,10,10-D4-arachidonic acid and esters thereof; 7-¹³C-7,7,13,13-D4-arachidonic acid and esters thereof, 7-¹³C-10,10,13,13-D4-arachidonic acid and esters thereof, 7-¹³C-7,7,10,10,13,13-D6-arachidonic acid and esters thereof; and combinations of these arachidonic species thereof. In each of these species, C7 is isotopically modified with ¹³C, and can additionally or alternatively have ¹³C at the C10 and/or C13 positions. Combining deuteration and ¹³C substitution at bis-allylic sites can result in larger protective effects, such as for example 7,7,10,10,13,13-D6, 7,10,13-¹³C3-arachidonic acid and esters thereof. In one embodiment, a method for preventing, reducing, or treating inflammation associated with tissues where AA is the dominant PUFA comprises administering 7,7,10,10,13,13-D6-arachidonic acid.

Methods for synthesizing site-specific isotopically modified essential PUFAs have been previously described. For example, a polyacetylene synthesis approach involves the assembly of C20 chain using iterative C-alkylation of propargyl alcohol dianion with electrophilic propargyl bromide or tosylate. (See, e.g., Fomich, M., et al., “Full Library of (Bis-allyl)-deuterated Arachidonic Acids: Synthesis and Analytical Verification,” Chemistry Select, 1, 4758-4764 (2016), which is hereby incorporated by reference.) The resulting tetrayne product is then partially hydrogenated to tetraene and purified chromatographically. The latter approach provides a convenient way to the deuterated arachidonic acids, as it allows using D2-propargyl alcohol instead of propargyl alcohol for creation of CD₂-group in the bis-allylic position when needed.

In certain embodiments, the D-AA comprising compositions of the present disclosure are administered during a treatment period, wherein the total amount of deuterated polyunsaturated fatty acid or fatty acid ester administered to or ingested by the patient is at least 1%, 2%, 3%, 4%, 5%, 10%, 15%, 20%, or 25% of the total amount of PUFA and/or ester thereof delivered and/or ingested into the patient's body such that over the treatment period the body of the patient incorporates significant amounts of deuterated polyunsaturated fatty acid or fatty acid ester and non-deuterated polyunsaturated fatty acid or fatty acid ester, such that the amount of deuterated polyunsaturated fatty acid or fatty acid ester in the patient's body is sufficient to reduce or prevent inflammation in the target tissue in the patient's body. In one embodiment, the total amount of D-PUFA or ester thereof that is administered (ingested or otherwise delivered) is at least 5% of the total amount of polyunsaturated fatty acid or fatty acid ester administered. In another embodiment, the total amount of D-PUFA or ester thereof that is administered is in the range of about 5% to about 75% of the total amount of polyunsaturated fatty acid or fatty acid ester delivered to the patient's body, In one embodiment, the deuterated fatty acid or ester thereof comprises between about 5% and 50%, about 5% and 30%, about 10% and 50%; and about 10% and 30%.

The delivery of the PUFAs, PUFA mimetics, PUFA pro-drugs, and triglycerides containing PUFAs and/or PUFA mimetics could be through a modified diet. Alternatively, the PUFAs, PUFA mimetics, PUFA pro-drugs, and triglycerides containing PUFAs and/or PUFA mimetics can be administered as foods or food supplements, on their own or as complexes with ‘carriers’, including, but not limited to, complexes with albumin.

Other methods of delivering the reinforced PUFAs or their precursors, such as methods typically used for drug delivery and medication delivery, can also be employed. These methods include, but are not limited to, peroral delivery, topical delivery, transmucosal delivery such as nasal delivery, nasal delivery through cribriform plate, intravenous delivery, subcutaneous delivery, inhalation, or through eye drops. Targeted delivery methods and sustained release methods, including, but not limited to, the liposome delivery method, can also be employed.

It is contemplated that the isotopically modified compounds described herein may be administered over a course of time, in which the cells and tissues of the subject will contain increasing levels of isotopically modified compounds over the course of time in which the compounds are administered.

Compositions containing the active ingredient may be in a form suitable for oral use, for example, as tablets, troches, lozenges, aqueous or oily suspensions, oil-in-water emulsions, dispersible powders or granules, emulsions, hard or soft capsules, or syrups or elixirs. Such compositions may contain excipients such as bulking agents, solubilization agents, taste masking agents, stabilizers, coloring agents, preservatives and other agents known to those ordinarily skilled in the art of pharmaceutical formulation. In addition, oral forms may include food or food supplements containing the compounds described herein. In some embodiments supplements can be tailor-made so that one type of PUFA, such as omega-3 or omega-6 fatty acids can be added to food or used as a supplement depending on the dominant fat that the food or the subject's diet contains.

In some embodiments, compounds are dosed at the following amounts (mg of active ingredient, i.e., isotopically-modified PUFAs per kg weight of subject): about 0.01 mg/kg to about 1000 mg/kg, about 0.1 mg/kg to about 100 mg/kg, and/or about 1 mg/kg to about 10 mg/kg. In other embodiments, compounds are dosed at about: 0.01, 0.1, 1.0, 5.0, 10, 25, 50, 75, 100, 150, 200, 300, 400, 500, and/or 1000 mg/kg.

While the present disclosure has been described with reference to the specific embodiments thereof, it should be understood by those skilled in the art that various changes may be made and equivalents may be substituted without departing from the true spirit and scope of the present disclosure. This includes embodiments which do not provide all of the benefits and features set forth herein. In addition, many modifications may be made to adapt a particular situation, material, composition of matter, process, process step or steps, to the objective, spirit and scope of the present disclosure. All such modifications are intended to be within the scope of the claims appended hereto.

### EXAMPLES

The present disclosure is further understood by reference to the following examples, which are intended to be purely exemplary of this invention. The present disclosure is not limited in scope by the exemplified embodiments, which are intended as illustrations of single aspects of this disclosure only. Any methods that are functionally equivalent are within the scope of this disclosure. Various modifications of this invention in addition to those described herein will become apparent to those skilled in the art from the foregoing description and accompanying figures. Such modifications fall within the scope of the appended claims. In these examples, the following terms are used herein and have the following meanings. If not defined, the abbreviation has its conventional medical meaning.

### Example 1: Administration of D-ARA in a Mouse Model of Lung Inflammation

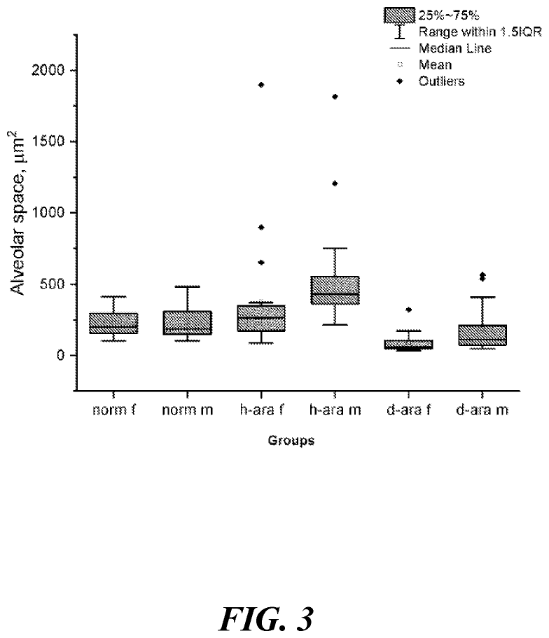

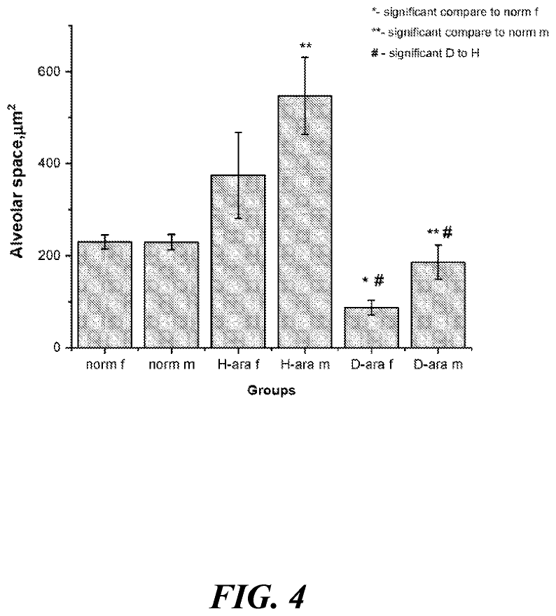

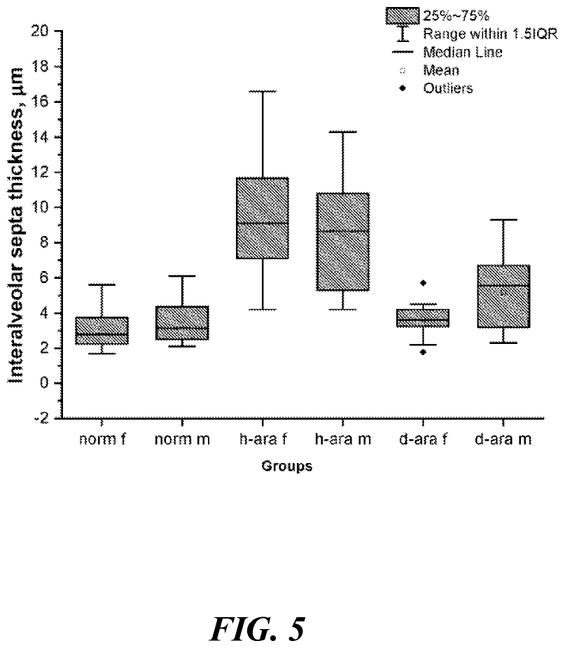

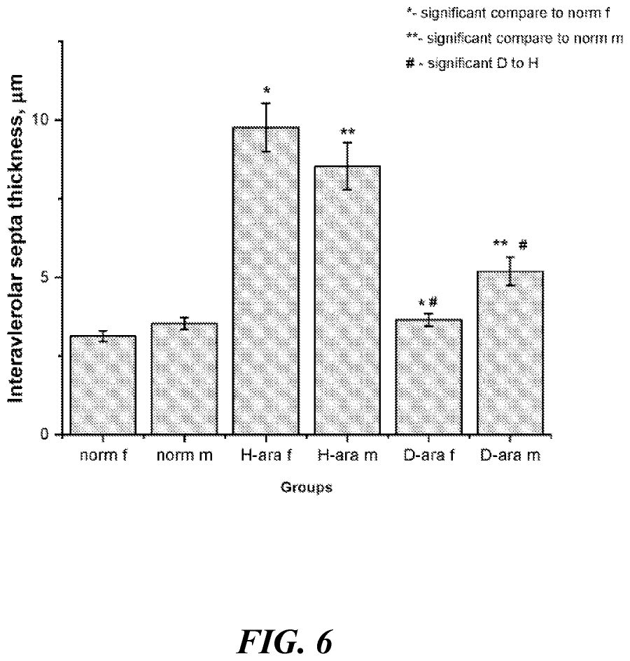

LPS treatment promotes inflammation through various mechanisms including secretion of pro-inflammatory cytokines, eicosanoids and induction of ROS. Following induction by the intranasal administration of LPS, an acute lung inflammation quickly follows. In mice receiving a 6-week course of dietary (H) ARA followed by single intranasal administration of LPS, the thickness of the interalveolar septa was significantly increased, which is likely associated with stronger edema and inflammatory infiltration. Such a pronounced inflammatory infiltration of the interalveolar septa contributed to their destruction and more frequent formation of emphysema foci in this group. At the same time, lungs of mice that received the D-form were characterized by decrease in alveoli lumens, which may be associated with a lower (compared with the H-form) frequency of emphysematous transformation of the lungs. (See FIGS. 3-6.)

The degree of alveolar lumen area changes in both groups of animals depended on gender. This indicator, regardless of the form of acid (H or D), in females was significantly lower than in males. Besides of this among the mice that received the D-form, the severity of changes in thickness of interalveolar septa was significantly higher in males compared to females, correlating well with human cases (females less affected). In general, the data obtained indicate a lesser degree of inflammatory lesion of the lungs after course of D-form compare to H-form. Preliminary data (not shown) also indicates that D6-ARA (7,7,10,10,13,13-D6-arachidonic acid) provides a better therapeutic effect as compared to other deuterated essential PUFAs.

Table 1. below summarizes the data underlying FIGS. 3-6.

TABLE 1

| | Group, gender | | | | | | |
|---|---|---|---|---|---|---|---|
| | | | H-ara + | H-ara + | D-ara + | D-ara + | |
| | Control, | Control, | LPS, | LPS, | LPS, | LPS, | |
| Morphological | males | females | males | females | males | females | Significance |
| criterion | 1 | 2 | 3 | 4 | 5 | 6 | Criterion, p |
| Alveolar lumen | 185.35 | 201.15 | 431.1 | 263.7 | 112.1 | 57.4 | pi-2 = 0.7327 pi- |
| area (25%; 75%), | (149.45; 308.70) | (156.00; 296.30) | (362.75; 554.1) | (173.85; 348.45) | (73.1; 209.95) | (46.15; 104.4) | 3 = 0.0000 p2- |
| ₚᵢₙ2 | | | | | | | 4 = 0.2068 |
| | | | | | | | P₁_{_{—}}₅ = 0.0225 |
| | | | | | | | p2.6 = 0.0000 |
| | | | | | | | p3-5 = 0.0000 |
| | | | | | | | pa-6 = 0.0000 |
| Partition thickness | p_{μm} ²8.65 3.15 | 2.8 | (5.3; 10.8) | 9.1 | 5.55 | 3.6 | pi-2 = 0.1284 |
| Me (25%; 75%), | (2.5; 4.35) | (2.25; 3.75) | | (7.1; 11.65) | (3.2; 6.7) | (3.25; 4.2) | pi-3 = 0.0000 |
| | | | | | | | p2-4 = 0.0000 |
| | | | | | | | pi⁵ =^{0.0011} p2- |
| | | | | | | | 6 = 0.0473 p3- |
| | | | | | | | 5 = 0.0018 pa- |
| | | | | | | | 6 = 0.0000 |

Oral data from human dosing in ongoing clinical trials show an impressive safety record for a similar D-PUFA drug which is a metabolic precursor to ARA. The expected safety of a D-PUFA drug combined with the convenience of oral dosing of D-PUFA gel caps warrant further studies of D-PUFAs as an approach for anti-inflammatory, and possibly preventative, therapy against COVID-19 induced cytokine storm and thrombosis events. Other PUFA emulsions such as IntraLipid are formulated in emulsions and dosed I.V. at multiple grams per day safely. One could imagine such a formulation could enable rapid, bolus dosing of the drug upon treatment onset, followed up by lipid gel cap oral dosing of D-ARA as a continuing therapy. These data present a rationale for further research on deuterated PUFA technology as an approach to COVID inflammation-associated therapy and other inflammation-associated conditions.

### Example 2: In Vitro Assay for Inflammation

In vitro cell-based assays for inflammation are well known in the art. These assays include such examples as e-selectin (also named Endothelial Leukocyte Adhesion Molecule or ELAM) and C-reactive protein (CRP). The ELAM assay can measure in vitro activity of test compounds in reducing expression of ELAM in activated endothelial cells.

For example, activated endothelial cells are created by adding known activators such as lipopolysaccharides, TNF, or IL-1(3 to rat intestinal MVEC cells. Activated cells are incubated with an isotopically modified PUFA such as a deuterated PUFA (“D-PUFA”) (0.01, 0.1, 1.0, 10.0, or 100 μM of D-PUFA and 1:1 or different ratio combinations of D-PUFA mixtures, e.g. D2-LA, D4-ALA, et al., see e.g. species that fall within Formula (1) supra) or “H-PUFA” (H-PUFA means not deuterated or tritiated) (0.01, 0.1, 1.0, 10.0, or 100 μM of LA, ALA, et al., and corresponding ratio combinations of non-deuterated mixtures) for 24, 48, and 72 hours. Activated cells are known to produce ELAM, which can be measured using, for example, an E-selectin monoclonal antibody-based ELISA assay. D-PUFA treated cells are expected to produce lower amounts of ELAM as compared to cells treated with H-PUFA.

Similarly, a CRP assay can be used to measure the in vitro activity of test compounds in reducing expression of CRP in Human Hep3B epithelial cells. For example, activated epithelial cells are created by adding known activators such as lipopolysaccharides, TNF, or IL-1β to Human Hep3B epithelial cells. Activated cells are incubated with D-PUFA (0.01, 0.1, 1.0, 10.0, or 100 μM), and different ratio combinations of different D-PUFAs) or H-PUFA (0.01, 0.1, 1.0, 10.0, or 100 μM of LA, ALA, and corresponding combinations of H-PUFAs) for 24, 48, and 72 hours. Activated cells are known to produce CRP, which can be measured with a CRP ELISA assay. D-PUFA treated cells are expected to produce lower amounts of CRP as compared to cells treated with H-PUFA.

### Example 3: In Vivo Assay for Inflammation

In vivo evaluation of anti-inflammatory activity can be determined by well characterized assays measuring Carrageenan-Induced Paw Edema and by Mouse Ear Inflammatory Response to Topical Arachidonic Acid. (See Gabor, M., Mouse Ear Inflammation Models and their Pharmacological Applications, 2000, which is incorporated herein by reference). Carrageenan-Induced Paw Edema is a model of inflammation that measures time-dependent edema formation following carrageenan administration into the intraplantar surface of a rat paw. Groups (8-9 animals/group) of 8-week old Wistar albino rats are supplemented with D-PUFA (see, e.g., species of Formula (1)) (0.01, 0.1, 1.0, 10.0, and 100 mg/kg), and different combinations of D-PUFAs (e.g., 1:1 ratio of two types of D-PUFAs) or H-PUFA (0.01, 0.1, 1.0, 10.0, and 100 mg/kg) and different combinations of H-PUFA corresponding to the different combination of D-PUFA, as the only PUFA source for a period of 8 weeks. Following the supplementation period, the rats are lightly anaesthetized under isofluorane and receive a subplantar injection of 50 μL saline containing 1% w/v carrageenan. Paw volumes are determined using a water plethysmometer and compared to paw volume prior to carrageenan administration. Volumes are measured at 0.5, 1, 2, 3, 4, and 5 hr. Edema can be calculated as the increase in paw volume divided by the starting paw volume. Rats supplemented with D-PUFA are expected to have lower levels of edema as compared to Rats supplemented with H-PUFA.

Additionally, the application of arachidonic acid (ARA) to the ears of rats is known to produce immediate vasodilation and erythema, followed by the abrupt development of edema, which should be maximal at 40 to 60 min. The onset of edema is believed to coincide with the extravasations of protein and leukocytes. After one hour the edema should wane rapidly and the inflammatory cells should leave the tissue so that at 6 hours the ears will have returned to near normal. Groups (8-9 animals/group) of 8-week old male rats are supplemented with D-PUFA (see, e.g., species of Formula (1)) (0.01, 0.1, 1.0, 10.0, and 100 mg/kg of D-PUFA, and different combinations of D-PUFAs (e.g., 1:1 ratio of two types of D-PUFAs) or H-PUFA (0.01, 0.1, 1.0, 10.0, and 100 mg/kg) and different combinations of H-PUFA corresponding to the different combination of D-PUFA, as the only PUFA source for a period of 8 weeks. Following the supplementation period, arachidonic acid is applied to the ears of the rats and vasodilation, erythema and edema is measured as a function of time. Rats supplemented with D-PUFA are expected to have lower levels of edema as compared to Rats supplemented with H-PUFA.

### Example 4: Collagen-Induced Arthritis Model

Groups (8-9 animals/group) of DBA/1 mice 8-10 weeks of age are supplemented with D-PUFA (see, e.g., species of Formula (1)) (0.01, 0.1, 1.0, 10.0, and 100 mg/kg of D-PUFA, and different combinations of D-PUFAs (e.g., 1:1 ratio of two types of D-PUFAs) or H-PUFA (0.01, 0.1, 1.0, 10.0, and 100 mg/kg) and different combinations of H-PUFA corresponding to the different combination of D-PUFA, as the only PUFA source for a period of 8 weeks. Mice are then injected with 100 pg bovine type II collagen in Freund's complete adjuvant (FCA) intradermally at the base of the tail and monitored by daily examination for the onset of disease, which is recorded. D-PUFA treated mice are expected to have a delayed onset, it any onset, of arthritis symptoms as compared to H-PUFA treated mice.

Alternatively, forty-five DBA/1 mice 8-10 weeks of age are injected with 100 pg bovine type II collagen in Freund's complete adjuvant (FCA) intradermally at the base of the tail and monitored by daily examination for the onset of disease, which is recorded. At the first appearance of clinical evidence of arthritis, mice are divided randomly into one of three treatment groups: 1) control; 2) D-PUFA (0.01, 0.1, 1.0, 10.0, and 100 mg/kg), and 1:1 or different combinations of different species of D-PUFA) treated; or 3) H-PUFA (0.01, 0.1, 1.0, 10.0, and 100 mg/kg), and 1:1 or different combinations of H-PUFA corresponding in type to D-PUFA combinations. The severity of arthritis in the affected paw is graded according to an established score system as follows: 0 (normal joint), 1 (mild/moderate visible edema and swelling), 2 (severe edema with distortion of paw and joint) and 3 (deformed paw or joint with ankylosis). The sum of the scores for all four paws in each mouse is used as an arthritis index (maximum score/mouse=12) to represent overall disease severity and progression in the animal. Animals re clinically assessed for disease five times per week until ten weeks after disease onset, and paw measurements are made three times per week. Arthritic paws without signs of disease at any time following treatment are considered in remission. All mice are pre-bled prior to the start of the trial, subsequently at onset of arthritis, two weeks post onset, four weeks post onset and at the completion of the trial. Sera obtained from each group is stored at 80° C. until needed. ELISA assays are performed to determine total anti-collagen antibody levels in mouse CIA. D-PUFA treated animals are expected to have reduced signs of arthritis as compared to H-PUFA treated animals and control animals.

While preferred embodiments of the present disclosure have been shown and described herein, it will be obvious to those skilled in the art that such embodiments are provided by way of example only. It is not intended that the disclosure be limited by the specific examples provided within the specification. While the disclosure has been described with reference to the aforementioned specification, the descriptions and illustrations of the embodiments herein are not meant to be construed in a limiting sense. Numerous variations, changes, and substitutions will now occur to those skilled in the art without departing from the disclosure. Furthermore, it shall be understood that all aspects of the disclosure are not limited to the specific depictions, configurations or relative proportions set forth herein which depend upon a variety of conditions and variables. It should be understood that various alternatives to the embodiments of the disclosure described herein may be employed in practicing the disclosure. It is therefore contemplated that the present disclosure shall also cover any such alternatives, modifications, variations or equivalents. It is intended that the following claims define the scope of the disclosure and that methods and structures within the scope of these claims and their equivalents be covered thereby.

1. A method for treating, preventing or reducing inflammation of a subject, the method comprising administering to the subject a composition comprising one or more compounds of Formula (1): 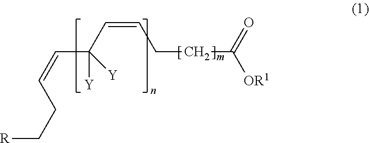
wherein: n=1-5; m=1-10; R=H, C₃H₇; R¹=H, alkyl, or a cation; Y=hydrogen, deuterium, or tritium so long as there is at least one Y that is substituted with a deuterium or tritium; and each bis-allylic carbon is either ¹²C or ¹³C.

2. The method of claim 1, wherein the one or more compounds of Formula (1) comprise one or more of: 11-D-linoleic acid; 11,11-D2-linoleic acid; 11-D-linolenic acid; 11,11-D2-linolenic acid; 14-D-linolenic acid; 14,14-D2-linolenic acid; 11,14-D2-linolenic acid; 11,11,14,14-D4-linolenic acid; 7,7-D2-arachidonic acid; 10,10-D2-arachidonic acid; 13,13-D2-arachidonic acid; 7,7,10,10-D4-arachidonic acid; 7,7,13,13-D4-arachidonic acid; 10,10,13,13-D4-arachidonic acid; 7,7,10,10,13,13-D6-arachidonic acid; 7,7-D2-eicosapentaenoic acid; 10,10-D2-eicosapentaenoic acid; 13,13-D2-eicosapentaenoic acid; 16,16-D2-eicosapentaenoic acid; 7,7,10,10-D4-eicosapentaenoic acid; 7,7,13,13-D4-eicosapentaenoic acid; 7,7,16,16-D4-eicosapentaenoic acid; 10,10,13,13-D4-eicosapentaenoic acid; 10,10,16,16-D4-eicosapentaenoic acid; 13,13,16,16-D4-eicosapentaenoic acid; 7,7,10,10,13,13-D6-eicosapentaenoic acid; 7,7,10,10,16,16-D6-eicosapentaenoic acid; 7,7,13,13,16,16-D6-eicosapentaenoic acid; 10,10,13,13,16,16-D6-eicosapentaenoic acid; 7,7,10,10,13,13,16,16-D8-eicosapentaenoic acid; 6,6-D2-docosahexaenoic acid; 9,9-D2-docosahexaenoic acid; 12,12-D2-docosahexaenoic acid; 15,15-D2-docosahexaenoic acid; 18,18-D2-docosahexaenoic acid; 6,6,9,9-D4-docosahexaenoic acid; 6,6,12,12-D4-docosahexaenoic acid; 6,6,15,15-D4-docosahexaenoic acid; 6,6,18,18-D4-docosahexaenoic acid; 9,9,12,12-D4-docosahexaenoic acid; 9,9,15,15-D4-docosahexaenoic acid; 9,9,18,18-D4-docosahexaenoic acid; 12,12,15,15-D4-docosahexaenoic acid; 12,12,18,18-D4-docosahexaenoic acid; 15,15,18,18-D4-docosahexaenoic acid; 6,6,9,9,12,12-D6-docosahexaenoic acid; 6,6,9,9,15,15-D6-docosahexaenoic acid; 6,6,9,9,18,18-D6-docosahexaenoic acid; 9,9,12,12,15,15-D6-docosahexaenoic acid; 9,9,12,12,18,18-D6-docosahexaenoic acid; 12,12,15,15,18,18-D6-docosahexaenoic acid; 6,6,15,15,18,18-D6-docosahexaenoic acid; 9,9,15,15,18,18-D6-docosahexaenoic acid; 6,6,12,12,15,15-D6-docosahexaenoic acid; 6,6,12,12,18,18-D6-docosahexaenoic acid; 6,6,9,9,12,12,15,15-D8-docosahexaenoic acid; 6,6,9,9,12,12,18,18-D8-docosahexaenoic acid; 6,6,9,9,15,15,18,18-D8-docosahexaenoic acid; 6,6,12,12,15,15,18,18-D8-docosahexaenoic acid; 9,9,12,12,15,15,18,18-D8-docosahexaenoic acid; 6,6,9,9,12,12,15,15,18,18-D10-docosahexaenoic acid; and salts or esters thereof.

3. The method of claim 1, wherein the inflammation of the subject comprises an inflammation-related disorder associated with increased levels in ROS and/or lipid peroxidation in cells.

4. The method of claim 3, wherein the inflammation-related disorder associated with increased levels in ROS and/or lipid peroxidation in cells is: asthma, rheumatoid arthritis, juvenile chronic arthritis, osteoarthritis, myositis, Crohn's disease, gastritis, colitis, ulcerative colitis, inflammatory bowel disease, proctitis, pelvic inflammatory disease, systemic lupus erythematosus, rhinitis, conjunctivitis, scleritis, chronic inflammatory polyneuropathy, Lyme disease, psoriasis, dermatitis, eczema, autoimmune disorders, atherosclerosis, or COVID-19.

5. A method for treating, preventing or reducing inflammation of a subject, the method comprising administering to the subject a composition comprising an arachidonic acid or ester thereof that has at least one deuterium at a bis-allylic position.

6. The method of claim 5, wherein the inflammation of the subject involves eicosanoids as pro- or anti-inflammatory signals.

7. The method of claim 6, wherein the inflammation of the subject relates to fever, allergy, lung disease, respiratory disorders, COVID-19-related cytokine storm, rheumatoid arthritis, lupus, COPD, asthma, ulcerative colitis, Crohn's disease, or glomerulonephritis.

8. The method of claim 5, wherein the inflammation of the subject is in the lungs.

9. The method of claim 8, wherein the inflammation in the lungs is due to COVID-19.

10. The method of claim 9, wherein the inflammation relates to thrombotic complications for COVID-19 patients.

11. The method of claim 5, wherein the subject suffers from thrombosis.

12. The method of claim 5, wherein the inflammation is associated with lipid peroxidation.

13. The method of claim 5, wherein the at least one deuterium is at the C7 bis-allylic position.

14. The method of claim 5, wherein the composition comprises one or more of the following: 7,7-D2-arachidonic acid and esters thereof; 10,10-D2-arachidonic acid and esters thereof; 13,13-D2-arachidonic acid and esters thereof; 7,7,10,10-D4-arachidonic acid and esters thereof; 7,7,13,13-D4-arachidonic acid and esters thereof; 10,10,13,13-D4-arachidonic acid and esters thereof; 7,7,10,10,13,13-D6-arachidonic acid and esters thereof; and combinations thereof.

15. The method of claim 5, wherein the composition comprises one or more of the following: 7,7-D2-arachidonic acid and esters thereof 7,7,10,10-D4-arachidonic acid and esters thereof; 7,7,13,13-D4-arachidonic acid and esters thereof; 7,7,13,13-D4-arachidonic acid and esters thereof; 7,7,10,10,13,13-D6-arachidonic acid and esters thereof and combinations thereof.

16. The method of claim 5, wherein the composition comprises 7,7,10,10,13,13-D6-arachidonic acid and esters thereof.
